(19) United States
(12) Patent Application Publication
(10) Pub. No.: US 2008/0262549 A1
(43) Pub. Date: Oct. 23, 2008

(54) METHODS AND SYSTEMS FOR DEPLOYING SPINOUS PROCESS CONSTRAINTS

(75) Inventors: Ian Bennett, San Francisco, CA (US); Colin Cahill, San Francisco, CA (US); Todd Alamin, Woodside, CA (US); Louis Fielding, San Carlos, CA (US); Hugues Malandain, Mountain View, CA (US); Manish Kothari, San Rafael, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Simpirica Spine, Inc., Redwood City, CA (US)

(21) Appl. No.: 12/106,049

(22) Filed: Apr. 18, 2008

Related U.S. Application Data
(63) Continuation-in-part of application No. 11/875,674, filed on Oct. 19, 2007.
(60) Provisional application No. 60/862,085, filed on Oct. 19, 2006.

(51) Int. Cl. A61B 17/70; A61B 19/00

(52) U.S. Cl. 606/263; 128/898; 606/246

(57) ABSTRACT

An exemplary method for constraining spinous processes to elastically limit flexion of a spinal segment comprises piercing an interspinous ligament to form a first penetration above an upper side of an upper spinous process and advancing a first end of a first tether through the first penetration. The interspinous ligament is pierced again to form a second penetration below a lower side of a lower spinous process and a second end of a second tether is advanced through the second penetration. Joining the first and second tethers together forms an extensible tether structure coupling the upper and lower spinous processes together while permitting extension therebetween. Adjusting the tether structure sets relative distance or angle between the upper and lower spinous processes to a target value.

### CROSS-REFERENCES TO RELATED APPLICATIONS

The present application is a continuation-in-part of U.S. application Ser. No. 11/875,674 (Attorney Docket No. 026398-000150US), filed Oct. 19, 2007, which claims the benefit of U.S. Provisional Patent Application No. 60/862,085 (Attorney Docket No. 026398-000100US), filed on Oct. 19, 2006. The entire contents of the above listed applications are incorporated herein by reference.

### BACKGROUND OF THE INVENTION

1. Field of the Invention

The present invention relates generally to medical methods and apparatus. More particularly, the present invention relates to methods and devices for restricting spinal flexion in patients having back pain or other spinal conditions.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine (FIG. 1). Pain tends to be exacerbated when patients put their lumbar spines into flexion (i.e. by sitting or bending forward) and relieved when they put their lumbar spines into extension (i.e. standing or arching backwards). Flexion and extension are known to change the mechanical loading pattern of a lumbar segment. When the segment is in extension (i.e. standing position), the axial loads borne by the segment are shared by the disc and the facet joints (approximately 30% of the load is borne by the facet joints). In flexion, however, the segmental load is borne almost entirely by the disc. Furthermore, when the segment is in flexion, the nucleus shifts posteriorly, changing the loads on the posterior portion of the annulus (which is innervated), likely causing its fibers to be subject to tension and shear forces. Segmental flexion, then, both increases the loads borne by the disc and causes them to be borne in a more painful way. Discogenic pain can be quite disabling, and for some patients, can dramatically affect their ability to work and otherwise enjoy their lives.

Patients with discogenic pain accommodate their syndrome by avoiding positions such as sitting, which cause their painful segment to go into flexion, and preferring positions such as standing, which maintain their painful segment in extension. One approach to reducing discogenic pain involves the use of a lumbar support pillow often seen in office chairs. Biomechanically, the attempted effect of the ubiquitous lumbar support pillow is also to maintain the painful lumbar segment in the less painful extension position. Another solution involves the use of an elastic tether structure coupled to the spinal segment. The tether structure can relieve pain by increasing passive resistance to flexion, mimicking the mechanical effect of postural accommodations that patients already use to provide relief.

Pain experienced by patients with discogenic low back pain can be thought of as flexion instability, and is related to flexion instability manifested in other conditions. The most prevalent of these is spondylolisthesis, a spinal condition in which abnormal segmental translation is exacerbated by segmental flexion. The device described here should as such also be useful for these other spinal disorders associated with segmental flexion, for which the prevention or control of spinal segmental flexion is desired.

Current treatment alternatives for patients diagnosed with chronic discogenic pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion may or may not also include instrumentation of the affected spinal segment including, for example, pedicle screws and stabilization rods. Fusion is not usually recommended for discogenic pain because it is irreversible, costly, associated with high morbidity, and of questionable effectiveness. Despite its drawbacks, however, spinal fusion for discogenic pain remains common due to the lack of viable alternatives.

An alternative method, that is not commonly used in practice, but has been approved for use by the United States Food and Drug Administration (FDA), is the application of bone cerclage devices that can encircle the spinous processes or other vertebral elements and thereby create a restraint to motion. Physicians typically apply a tension or elongation to the devices that applies a constant and high force on the anatomy, thereby fixing the segment in one position and allowing effectively no motion. The lack of motion allowed after the application of such devices is thought useful to improve the likelihood of fusion performed concomitantly; if the fusion does not take, these devices will fail through breakage of the device or of the spinous process to which the device is attached. These devices are designed for static applications and are not designed to allow for a dynamic elastic resistance to flexion across a range of motion. The purpose of bone cerclage devices and the other techniques described above is to almost completely restrict measurable motion of the vertebral segment of interest. This loss of motion at a given segment gives rise to abnormal loading and motion at adjacent segments leading eventually to adjacent segment morbidity.

Recently, a less invasive and potentially more effective treatment for discogenic pain has been proposed. A spinal implant has been designed which inhibits spinal flexion while allowing substantially unrestricted spinal extension. The implant is placed over one or more adjacent pairs of spinous processes and provides an elastic restraint to the spreading apart of the spinous processes which occurs during flexion. Such devices and methods for their use are described in U.S. Patent Publication No. 2005/02161017A 1, published on Sep. 29, 2005, and having common inventors with the present application, the entire contents of which are incorporated herein by reference.

As illustrated in FIG. 2, an implant 10 as described in the '017 publication, typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring or rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinous processes which provides a force that resists flexion. The force increases as the processes move further apart. Usually, the straps themselves will be essentially non-compliant so that the degree of elasticity or compliance may be controlled and provided solely by the compliance members 16.

The manner in which flexion is restricted with such an implant is controlled in part by the physical characteristics of the implant and in part by the way in which it is implanted in the patient. The physician controls the anterior-posterior location on the spinous processes at which the strap is placed. In a preferred embodiment, the physician can adjust the elasticity of the implant. In a preferred embodiment, the physician can further adjust the final size or tension of the implant; in particular, the physician can adjust the effective length of the implant. The effective length of the implant is the length of the portion of the implant that is engaged when the patient flexes. If the implant is a continuous structure as in FIG. 2, the effective length is the inner perimeter of the structure. The implant may be a structure that passes around one spinous process and is attached at two ends to a spinous process or sacrum, as described in U.S. patent application Ser. No. 11/777,366 (Attorney Docket No. 026398-000110US), filed on Jul. 13, 2007, and having common inventors with the present application, the entire contents of which are incorporated herein by reference. In this case, the effective length is the distance along the tether structure between the attachment points.

The manner in which flexion is restricted with such an implant can have a significant effect on the surgical outcome for a patient. If an implant intended to restrict flexion is deployed such that it applies too much tension to the spinous processes, the patient may develop complications such as facet arthropathy or lateral recess stenosis. If an implant intended to restrict flexion applies too little tension to the spinous processes, it will have little impact when the segment undergoes a small amount of flexion from the natural neutral position and hence the patient's pain may not be adequately relieved. The terms “neutral position,” “flexion” and “extension” will be defined in greater detail below.

This problem of balancing the need to deploy the implant such that it applies enough tension to the spinous processes to relieve pain but not so much tension that it causes complications is unique to this implant. Other structures deployed near or around the spinous processes such as those described by Bevan (U.S. Pat. No. 5,725,582) and Graf (U.S. Patent Publication No. 2004/0116927) are typically adjusted to be as tight as possible, either locking patients into extension or immobilizing the spinal segment in conjunction with a spinal fusion.

For these reasons, it would be desirable to provide methods and tools so that a physician can easily deploy the implant such that it applies an amount of tension on the spinous processes that is sufficient to relieve pain but not excessive. As such, the following invention relates to methods and tools for use in positioning and deploying an implant like that described in U.S. Patent Publication No. 2005/0216017A1.

2. Description of the Background Art

U.S. Patent Publication No. 2005/0216017A1 has been described above. Other patents and published applications of interest include: U.S. Pat. Nos. 4,966,600; 5,011,494; 5,092,866; 5,116,340; 5,282,863; 5,395,374; 5,415,658; 5,415,661; 5,449,361; 5,456,722; 5,462,542; 5,496,318; 5,540,698; 5,609,634; 5,645,599; 5,725,582; 5,902,305; Re. 36,221; 5,928,232; 5,935,133; 5,964,769; 5,989,256; 6,053,921; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; Published U.S. Patent Application Nos. 2002/0151978; 2004/0024458; 2004/0106995; 2004/0116927; 2004/0117017; 2004/0127989; 2004/0172132; 2005/0033435; 2005/0049708; 2006/0069447; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; and Published Foreign Application Nos. EP 0322334 A1; and FR 2 681 525 A1.

### BRIEF SUMMARY OF THE INVENTION

The present invention provides methods and tools for the deployment of spinal implants for restricting flexion of spinal segments in the treatment of discogenic pain and other spinal conditions, such as degenerative spondylolisthesis, where a physician may desire to control segmental flexion.

As used herein, “neutral position” refers to the position in which the patient's spine rests in a relaxed standing position. The “neutral position” will vary from patient to patient. Usually, such a neutral position will be characterized by a slight curvature or lordosis of the spine where the spine has a slight anterior convexity and slight posterior concavity. In some cases, the presence of the constraint of the present invention may modify the neutral position, e.g. the device may apply an initial force which defines a “new” neutral position having some extension of the untreated spine. As such, the use of the term “neutral position” is to be taken in context of the presence or absence of the device. As used herein, “neutral position of the spinal segment” refers to the position of a spinal segment when the spine is in the neutral position.

Furthermore, as used herein, “flexion” refers to the motion between adjacent vertebrae in a spinal segment as the patient bends forward. Referring to FIG. 1A, as a patient bends forward from the neutral position of the spine, i.e. to the right relative to a curved axis A, the distance between individual vertebrae L on the anterior side decreases so that the anterior portion of the intervertebral disks D are compressed. In contrast, the individual spinous processes SP on the posterior side move apart in the direction indicated by arrow B. Flexion thus refers to the relative movement between adjacent vertebrae as the patient bends forward from the neutral position illustrated in FIG. 1A.

Additionally, as used herein, “extension” refers to the motion of the individual vertebrae L as the patient bends backward and the spine extends from the neutral position illustrated in FIG. 1A. As the patient bends backward, the anterior ends of the individual vertebrae will move apart. The individual spinous processes SP on adjacent vertebrae will move closer together in a direction opposite to that indicated by arrow B.

Moreover, as used herein, the phrase “elastic resistance” refers to an application of constraining force to resist motion between successive, usually adjacent, spinous processes such that increased motion of the spinous processes results in a greater constraining force. The elastic resistance will, in the inventions described herein, inhibit motion of individual spinal segments by, upon deformation, generating a constraining force transmitted directly to the spinous processes or to one or more spinous process and the sacrum. The elastic resistance can be described in units of stiffness, usually in units of force per deflection such as Newtons per millimeter (N/mm). In some cases, the elastic resistance will generally be constant (within ±5%) over the expected range of motion of the spinous processes or spinous process and sacrum. In other cases, typically with elastomeric components, the elastic resistance may be non-linear, potentially varying from 33% to 100% of the initial resistance over the physiologic range of motion. Usually, in the inventions described herein the pre-operative range of motion of the spinous process spreading from the neutral or upright position to a maximum flexion-bending position will be in the range from 2 mm to 20 mm, typically from 4 mm to 12 mm. With the device implanted, the post-operative range of motion of the spinous process spreading from the neutral or upright position to a maximum flexion-bending position will be reduced and will usually be in the range from 1 mm to 10 mm, typically from 2 mm to 5 mm. Such spinous process spreading causes the device to undergo deformations of similar magnitude.

In a first aspect of the present invention, a method for constraining spinous processes to elastically limit flexion of a spinal segment comprises piercing an interspinous ligament to form a first penetration above an upper side of an upper spinous process and advancing first end of a first tether through the first penetration. Piercing the interspinous ligament again forms a second penetration below a lower side of a lower spinous process so that a second end of a second tether may be advanced through the second penetration. The first and second tethers are joined to form an extensible tether structure wherein the structure couples the upper and the lower spinous processes together while still permitting extension therebetween. Adjusting the tether structure sets a relative distance or angle between the upper and lower spinous processes to a target value.

In another aspect of the present invention, a method for treating degenerative spondylolisthesis comprises creating a pathway across a midline of the spinal segment or piercing an interspinous ligament to form a first penetration or pathway above an upper side of an upper spinous process and advancing a first end of a first tether through the first penetration or along the pathway. Creating a second pathway across the midline of the spinal segment, sometimes by piercing the interspinous ligament, forms a second penetration or pathway below a lower side of a lower spinous process so that a second end of a second tether may be advanced through the second penetration or along the second pathway. The first and second tethers are joined to form an extensible tether structure wherein the structure couples the upper and lower spinous processes together thereby elastically limiting flexion of a spinal segment containing the upper and lower spinous processes while permitting extension therebetween. The method also comprises decompressing the spinal segment and adjusting the tether structure so as to set relative distance or angle between the upper and lower spinous processes to a target value. Decompressing the spinal segment may include removing bone, disc or ligaments. In some cases, a defect or previous surgical intervention may make it unnecessary to pierce the interspinous ligament above an upper side of an upper spinous process or below a lower side of a lower spinous process. In these instances, creating the pathway may only require removing or displacing tissue in the area to clear and form the penetration or enlargement of an existing penetration.

In still another aspect of the present invention, a method for treating a spinal disorder comprises creating a pathway or piercing an interspinous ligament to form a first penetration above an upper side of an upper spinous process and advancing a first end of a first tether through the first penetration or along the pathway. Creating a second pathway through the interspinous ligament, sometimes by piercing it, forms a second penetration below a lower side of a lower spinous process so that a second end of a second tether may be advanced through the second penetration or along the second pathway. The first and second tethers are joined to form an extensible tether structure wherein the structure couples the upper and lower spinous processes together thereby elastically limiting flexion of a spinal segment containing the upper and lower spinous processes while permitting extension therebetween. The method also comprises measuring the tension in the tether structure and adjusting the tether structure so as to set the tension to a target value.

In either aspect of the present invention, the method may further provide the formation of a tether structure that provides a minimum and preferably no elastic resistance to lateral bending or rotation of the spinal segments. This is particularly true in the lumbar spine where the range-of-motion in rotation is usually limited to +/−3 degrees. The upper and lower spinous processes may be directly adjacent one another or there may be intermediate spinous processes in between. The methods further comprise advancing, pushing or pulling separate first and second tethers through the penetrations or along the pathways and joining more than one pair of ends to form the continuous structure. Sometimes the penetrations are also expanded. In one embodiment, the tethers are pushed through the penetrations as they are formed. The tethers may be advanced from the same side of the spinal segment midline or from opposite sides of the midline. When the first and second tethers are separate, additional ends of the tethers may be joined together to form the tether structure. In other embodiments, the first and second tethers may already be coupled together to form a continuous tether line and joining the first and second free ends forms the continuous tether structure. Another aspect of the present invention may further include passing a guidewire or guiding sheath along the path desired for the tether and using the guidewire or guiding sheath to direct the tether into position around the spinous processes. In all aspects of the present invention, the steps described for positioning the implant preferably minimally disrupt the muscles, tendons, and ligaments so as to preserve intact as much of the native anatomy as possible. In particular, the methods described will in all cases minimize disruption of the supraspinous ligament. Moreover, in most cases, the methods further comprise obtaining exposure to the preferred location for the implant without significantly disrupting the multifidus tendons. Furthermore, in cases in which a midline decompression is not performed, the methods further avoid disruption of the interspinous ligament between the coupled spinous processes.

In some patients, the geometry of the spinous process S1 on the sacrum may be such that the tether may not be adequately secured by passing it below the spinous process. In such instances, the methods may further comprise creating a hole in the sacrum and passing the tether structure through the hole, or inserting a sacral attachment member such as a hook or islet in the sacrum and passing the tether structure around or through the attachment member. In such instances, the methods may alternatively comprise inserting a screw into the sacrum and attaching the tether structure to the screw.

In some patients, the geometry of another spinous process may be such that the tether may not be adequately secured by passing it around the spinous process. For example, the spinous process may be defective or be intraoperatively damaged. In such instances, the methods may further comprise inserting a screw into the vertebral body and attaching the tether structure to the screw. In still other instances, a screw may be inserted into a pedicle and the tether structure attached to the screw.

In another aspect of the present invention, the methods may further comprise treating a spinous process. Treating may consist of creating a depression in the spinous process in which the band can rest. Such a depression could be creating by any means of removing soft tissue and bone, including sanding, grinding, drilling, or notching. Treating may alternatively comprise delivering a chemical or biological preparation to the spinous process, such as a preparation of stem cells, growth factors, adhesives, or a chemical coating. Such a preparation may promote or prevent growth of the spinous process into or around the tether structure. The methods herein described for treating the spinous process may help to improve the biological interaction between the tether structure and the spinous process, such that potential complications such as inflammation, wear, and cracking are minimized.

In one embodiment of the present invention, the tether structures are alone joined to form a full continuous structure. A portion of the tether structures may provide an elastic resistance to elongation in response to an elongation force which results from flexion of the spinal segments between the adjacent spinous processes and/or the sacrum. Often, the tether structures will include at least two compliance members positioned such that they will lie symmetrically on opposite sides of the spinous processes when implanted.

In another embodiment of the present invention, additional components may be joined with the tether structure to form the continuous structure. Such components could be compliance members, tension members, compression members, adjustment members, or attachment members. Often, at least two compliance members are joined and positioned as part of the continuous structure such that they will lie symmetrically on opposite sides of the spinous processes when implanted. The compliance members will typically be coupled to non-compliant and/or cable components of the tether structure so that it is the compliance members which provide most or all of the compliance or elasticity in the implants.

In some cases, it will be desirable to deploy such an implant across a spinal segment which does not have a sufficient inferior spinous process for retaining a continuous structure. In these cases, the invention may further comprise providing an islet or hole in the lower vertebra or sacrum. Alternatively, rather than join the ends of the tethers to form a continuous structure, two separate ends which extend from a structure that is already passed above a superior spinous process may be anchored to the adjacent vertebra or sacrum using screws, dowels, staples, or any of the techniques described above.

In a further aspect of the present invention, the methods include using images of the patient's spine to determine the appropriate positioning and tensioning of the implant. Various landmarks on the spinal segment such as the spinous processes may be measured in different stages of flexion, extension and the neutral state or standing to help adjust the implant to the predetermined distance. Because the implant is designed to restrict flexion of the treated spinal segment, the physician may perform lateral radiographs in neutral, flexion, and extension positions to help determine positioning and tensioning of the tether. Of particular interest, the physician may note the segmental angles or spinous process distances at the segment to be treated. In one aspect of the present invention, the method includes a lateral radiograph to determine the distance between the points at which an implant would be likely to attach to the bone. For example, the method may include measuring the distance from the edge along the top of the superior spinous process where the structure would likely rest to the edge along the bottom of the inferior spinous process where the structure would likely rest in a lateral radiograph in the standing position. Such distance, or any other corresponding measurement, could then be subsequently used during the surgery to provide guidance for the physician with respect to positioning and tensioning of the implant.

Because the manner in which flexion is restricted can have a significant effect on the surgical outcome for a patient, the method further provides steps for determining an ideal position along the spinous process at which to deploy the tether. This may typically include determining the position above or below the spinous process at which to pierce the interspinous ligament to create the penetration through which the tether will be advanced. In one embodiment, the method includes engaging a positioning guide against a preselected anatomical landmark and positioning the tether along an axis provided by the guide. In a preferred embodiment, the method includes engaging the guide against the base of the spinous process or against the lamina near the base of the spinous process and positioning the tether along an anterior-posterior axis defined by the guide, although naturally such a positioning guide could be engaged with other anatomical landmarks. The positioning guide may be provided with a feature for engaging with the tool that penetrates the interspinous ligament. In an alternative embodiment, a single tool may be capable of both positioning the targeted penetration site relative to an anatomical landmark and creating the penetration. Such a joint tool may have one blunt aspect which engages with the base of the spinous process and extends along an anterior-posterior axis along the edge of the spinous process and a second sharp aspect which can be deployed perpendicular to the anterior-posterior axis to create the penetration at the targeted position in the interspinous ligament next to the spinous process. The tether may also be positioned manually along the spinous process or it may be wrapped around the spinous process, sometimes at least 360 degrees.

In a further aspect of the present invention, the method includes adjusting the continuous structure such that the implant applies a desired amount of elastic resistance to separation of the spinous processes or applies a desired initial tension on the spinous processes or sets the implant to a desired size. In one embodiment, the method for adjusting the continuous structure includes changing the elastic resistance of the continuous structure. In a preferred embodiment, the change in elastic resistance is effected by changing compliance components in the continuous structure. Stiffer compliance components may be included in the continuous structure for patients in need of greater flexion resistance, and less stiff compliance components may be joined in the continuous structure for patients in need of less flexion resistance. In another embodiment, the compliance component itself may be intentionally pre-tensioned or pre-relaxed in order to change the elastic resistance of the continuous structure.

In a more typical embodiment, the method for adjusting the implant includes changing the effective length of the structure. For embodiments that are continuous loops, the effective length of the structure is typically the inner perimeter of the continuous loop. Although the discussion here and will focus on changes to the inner perimeter of a continuous loop, it is recognized that similar methods apply to changing the effective length of a tether structure that passes around one spinous process and is attached at two ends to a spinous process or sacrum, for which the effective length consists of the length of the structure from one fixed attachment point to the other. Similar methods apply to changing the effective length of tether structures with alternative configurations that may also used to provide elastic resistance to flexion of a spinal segment or segments.

The methods for changing the effective length of the structure comprise increasing or decreasing the length of the portion of the tether structure that is engaged when the patient flexes. For example, it may be desirable to decrease the effective length of the tether structure. Such decreases may be effected by removing a length of the tether structure from the continuous loop and optionally severing the excess material. This may be accomplished by changing the position at which components in the tether structure are attached to each other such that some portion of the tether structure which was previously engaged during flexion is subsequently outside of the inner perimeter of the structure. For example, if the continuous structure includes an attachment element that clamps one portion of the tether, the attachment could be loosened, more of the tether could be passed through the attachment to the outside of the loop, and the attachment could be tightened again to reform a continuous loop with a reduced inner perimeter and thus an implant with a smaller effective length. Such decreases may also be effected by swapping in and out components of the tether structure. Tether structure length and/or tension may also be adjusted post-operatively in order to optimize tether structure performance after it has been implanted.

In one aspect of the present invention, measurements from images of a patient's spine are used to identify the desired effective length of the tether structure. The tether structure effective length may thus be adjusted based on information from such images until the desired effective length is reached.

In another aspect of the present invention, the method includes selecting and adjusting the components of the implant outside of the body, such that the implant, when deployed and joined into a continuous structure, already consists of the desired effective length. In another aspect of the present invention, the tether is engaged with a fixture outside of the body and the effective length of the tether structure is adjusted on the fixture.

In still another aspect of the present invention, the method provides for adjusting the tether structure during the surgery until the desired effective length is reached. The methods and tools described include features that could aid the physician in determining when the tether structure has been adjusted to the desired effective length. In one embodiment, the tether includes visual indicators of the length, which might be colored regions of the tether or marks on the tether or other components of the tether structure. Alternative components such as strain gages, force gages or digital readouts in the implant or the tool could alternatively indicate the length or tension in the tether structure. A force gage may be used to measure the force required to resist flexion. In one aspect of the present invention, the indicators are visible on x-ray or MRI, allowing the physician to use imaging to intraoperatively determine the effective length when the patient is in multiple positions. The tether structure may also be adjusted to set a relative angle between upper and lower spinous processes to a predetermined value, such as that seen in the neutral state or in the standing state. In some embodiments, it may be desirable to tighten the tether structure over the spinous processes so that a relatively low finite force is applied even before flexion of a spinal segment from a neutral position. In still other embodiments, adjusting the length of the tether structure or initial tension applied by the tether structure subsequently reduces translation of the upper spinous process relative to the lower spinous process along an anterior-posterior axis due to shear forces in the spinal segment.

Of particular importance are methods and tools that help the surgeon avoid unwanted slack in the tether structure. Such slack can consist of extra material that the surgeon fails to account for and that causes the actual effective length to be greater than the desired effective length. In one aspect, the tools include a tensioning block or spacer that is temporarily placed between the spinous processes across the spinal segment, such that the tether structure can be tensioned against the spinous processes without the spinous processes moving into extension and without disrupting the interspinous ligament. The tensioning block is then removed once the tether is adjusted so that no implant remains between the spinous processes. As an alternative to a block between the spinous processes, a tool could clamp the superior spinous process and clamp the inferior spinous process from both sides of the midline and then hold the clamps at a fixed distance from each other while the tether structure is tightened against the spinous processes. Other means for holding the spinous processes at a fixed distance from each other while the tether structure is tightened against them are also possible.

Systems according to the present invention include implants and tools. In one embodiment, such systems include at least one tether, a piercing tool having a tissue-penetrating distal tip and an anchor for releasably attaching an end of the tether; wherein the piercing tool is adapted to be advanced in an anterior direction toward the interspinous ligament and laterally so that the tissue-penetrating tip can be pierced through the ligament to push or pull the attached tether through the resulting penetration. Such systems may further include a tool for positioning the penetrations in the interspinous ligament at a targeted region along an anterior-posterior axis of a spinous process. Such systems often will further include an adjustment tool for adjusting the effective length of the tether structure. In addition, such systems often will further include stabilizing tools for maintaining the position of the implant and/or the spinous processes while the adjusting tool engages with the tether structure to adjust the effective length of the tether structure.

### BRIEF DESCRIPTION OF THE DRAWINGS

FIG. 1 is a schematic diagram illustrating the lumbar region of the spine.

FIG. 1A a schematic illustration showing a portion of the lumbar region of the spine taken along a saggital plane.

FIG. 2 illustrates a spinal implant of the type described in US 2005/0216017A1.

FIGS. 3A-3D illustrate additional tissue surrounding the spinous processes.

FIGS. 4A-4D illustrate an exemplary embodiment of the method for delivering an implant consisting of two tether structures with attached compliance members joined to form a single tether structure and adjusted to apply the targeted amount of tension to the spinous processes.

FIGS. 5A-5B illustrate the use of different number of tether structures to couple varying number of spinous processes together.

FIG. 6 is illustrates an exemplary embodiment of a stabilizing tool that maintains the position of the spinous processes to prevent them from moving towards each other when tension is applied to the tether structure during the adjustment process.

FIG. 7 is an exemplary embodiment of an adjustment tool used to change the effective length of the band.

FIGS. 8A-8D illustrate several embodiments of tension control mechanisms.

FIG. 9 is a schematic illustration of a positioning tool that is deployed along the spinous process to determine appropriate placement of the tether structure.

FIG. 10 shows a curved band used to help maintain position of the tether structure on a spinous process.

FIGS. 11A-11B show how wrapping the tether structure around a spinous process helps maintain tether position.

FIGS. 12A-12B show bone removal from L4 during decompression.

FIGS. 13A-13B show bone removal from L5 during decompression.

FIGS. 14A-14B illustrate the use of a tether structure and decompression in the treatment of degenerative spondylolisthesis.

### DETAILED DESCRIPTION OF THE INVENTION

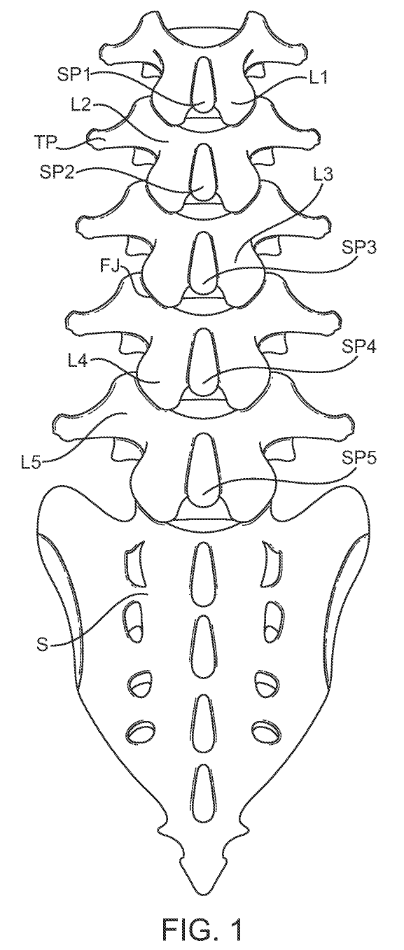

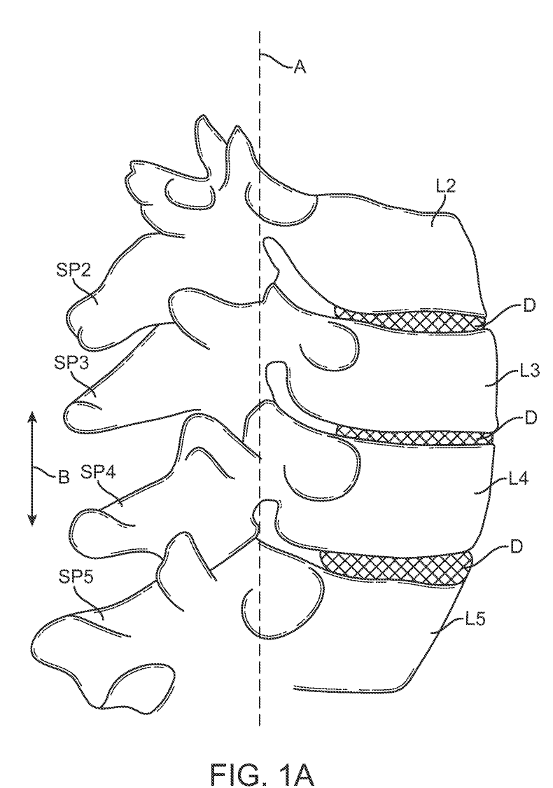

FIG. 1 is a schematic diagram illustrating the lumbar region of the spine including the spinous processes (SP), facet joints (FJ), lamina (L), transverse processes (TP), and sacrum (S). FIG. 1A is a schematic illustration showing a portion of the lumbar region of the spine taken along a saggital plane.

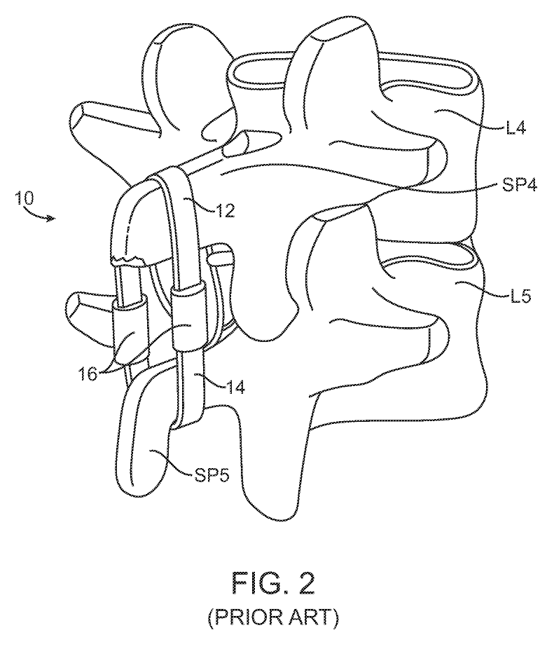

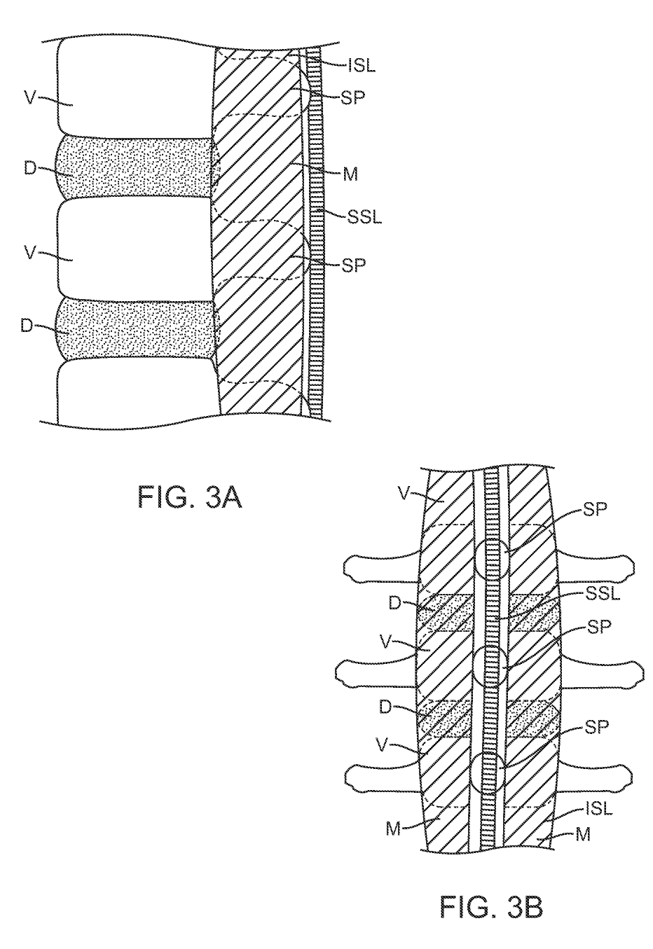

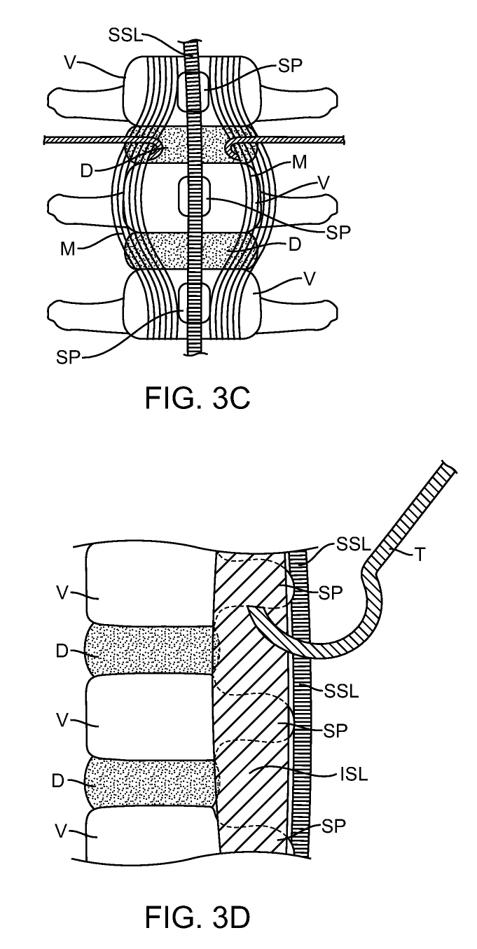

FIG. 3A is a side view of the lumbar region of the spine having discs D separating the vertebral bodies V. The supra spinous ligament SSL runs along the posterior portion of the spinous processes SP and the interspinous ligament ISL and multifidus tendon and muscle M run alongside of and attach to the spinous processes SP. FIG. 3B is a posterior view of FIG. 3A. FIG. 3C illustrates the lumbar region of the spine after an incision has been made through the patient's skin and the multifidus muscle and tendon M have been retracted to expose the spinous processes. In FIG. 3D a curved piercing tool may be used to access and pierce the interspinous ligament ISL while avoiding the supra spinous ligament SSL. This surgical approach is desirable since it keeps the supra spinous ligament intact and minimizes damage to the multifidus muscle and tendons and other collateral ligaments.

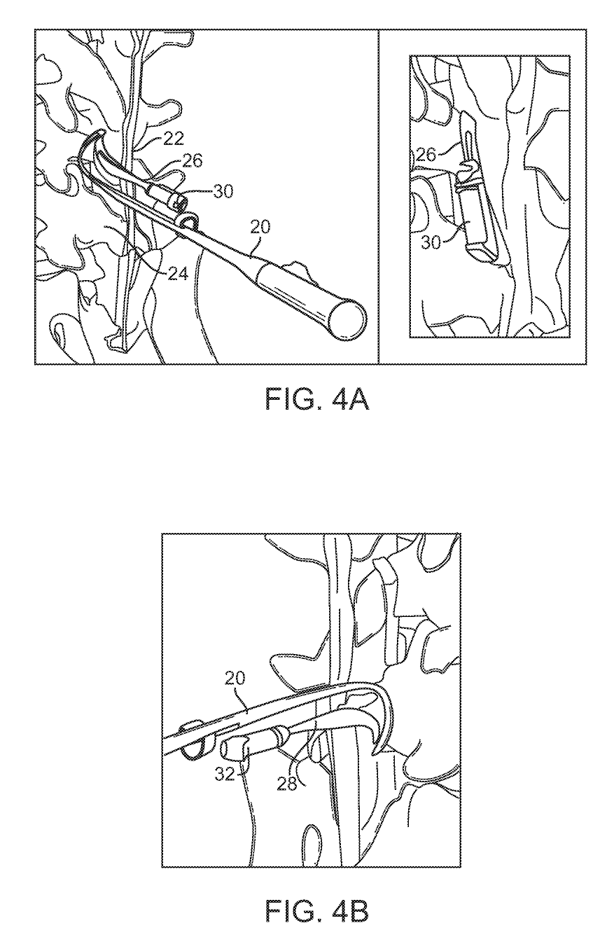

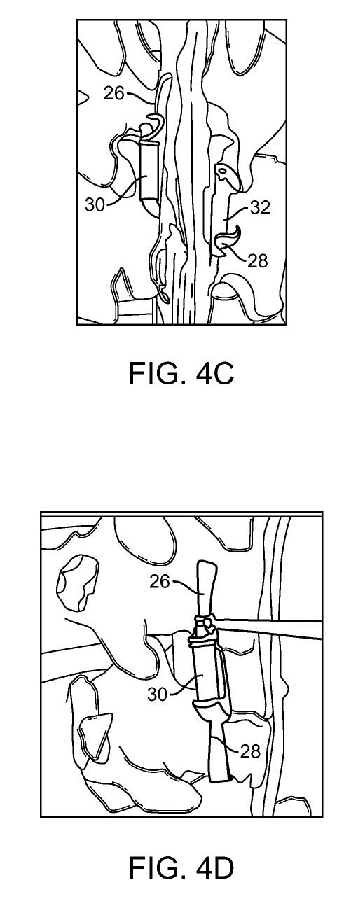

Referring now to FIGS. 4A-4D, a tool 20 suitable for use in accordance with the methods of the present invention is used to create penetrations above a superior spinous process 22 and below an inferior spinous process 24. Tethers, also referred to tether straps 26 and 28 including pre-attached compliance members 30 (FIG. 4A) and 32 (FIG. 4B) are advanced through the penetrations and joined (FIG. 4C) to form a continuous, multi-component tether structure. In some embodiments, tether straps 26, 28 are coupled together to form a continuous line and therefore only one end of each tether is joined to form the continuous tether structure. In other embodiments, the tether straps 26, 28 are separate tethers and the both ends of each of the two tether straps are joined to form the continuous tether structure. Other tether embodiments and joining methods are disclosed in U.S. Patent Publication No. 2008/0009866 (Attorney Docket No. 026398-000140US) and U.S. Provisional Patent Application No. 60/936,897 (Attorney Docket No. 026398-000400US), the entire contents of which are incorporated herein by reference. The tether straps 26 and 28 may be joined in situ or, in preferred embodiments, the tether straps 26 and 28 are joined outside of the patient's body. Typically, the tether structure is then adjusted (FIG. 4D) to increase or decrease its length, or to apply a desired amount of tension to the spinous processes, typically in the range from 0N to 30N, usually from 0N to 5N, assuming that the spinous processes are unconstrained during the tensioning process.

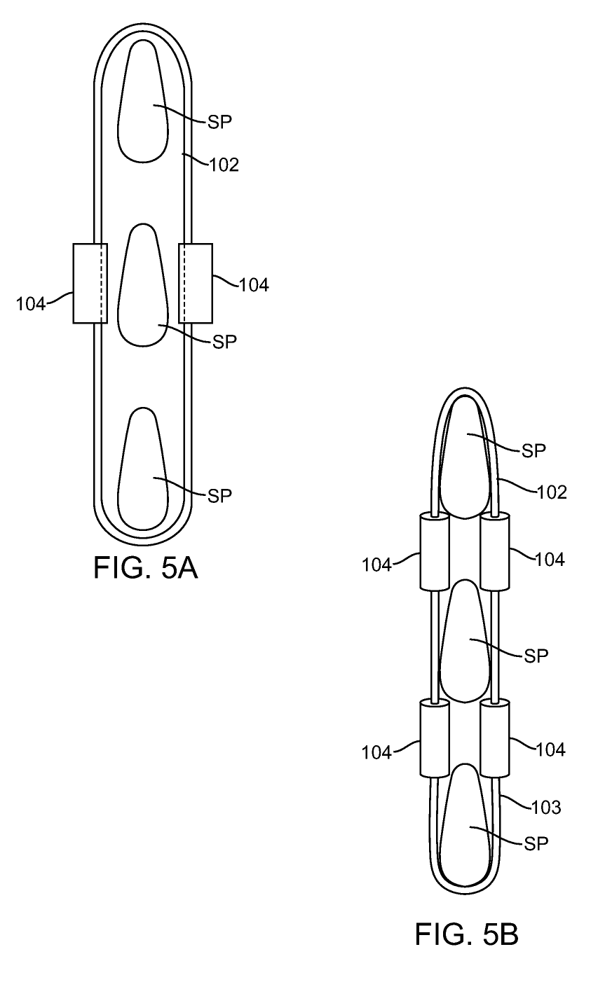

While FIGS. 2 and 4A-D illustrate a single tether structure wrapped around adjacent spinous processes without an intermediate spinous process therebetween, one will appreciate that a spinal segment having more than two spinous processes may be tethered together using one or more tether structures. For example, in FIG. 5A, a single tether 102 is used to couple two spinous processes SP together with an intermediate spinous process in between the upper and the lower spinous process SP. Compliance elements 104 are used to control extension between the spinous processes SP. FIG. 5B illustrates the use of two tether structures 102, 103 to couple a spinal segment containing three spinous processes together.

Referring now to FIG. 6, a tensioning block or spacer 34 or other stabilizing tool is optionally provided between the spinous processes 22, 24 to keep the spinous processes from extending while the tension or length of the tether structure is adjusted. Such stabilizing tools can allow the physician to remove unwanted slack from the deployed implant to achieve a targeted effective length and/or tension for the implant. The tether structure is adjusted while the stabilizing tool 34 prevents the spinous processes from extending, thus the targeted tension applied to the spinous processes during the adjustment procedure may be higher than the ranges described above for the method in which no such stabilizing tool is provided. Additionally, the spacer 34 may be placed between spinous processes without disrupting the interspinous ligament. Spacing between spinous processes may be adjusted to match the distance or an angle between spinous processes while the patient is in the neutral position or in the standing position.

Referring now to FIG. 7, the system and methods further comprise an adjustment tool 36 which engages with the tether structure 60 to change the effective length of the tether structure. Typically, an adjustment mechanism such as spool mechanism can be provided as part of the compliance members 30 and 32 to allow for tightening or loosening of the tethers 26 and 28. The adjustment tool 36 is rotated in order to tighten tether structure 60 and the excess tether material may optionally be cut or otherwise severed from the tether structure 60. Tension or length may be adjusted so that the distance between surrounding spinous processes is set to the distance seen while the patient is in the neutral or standing position or other position. Alternatively, tension or length may be adjusted to control an angle between the spinous processes.

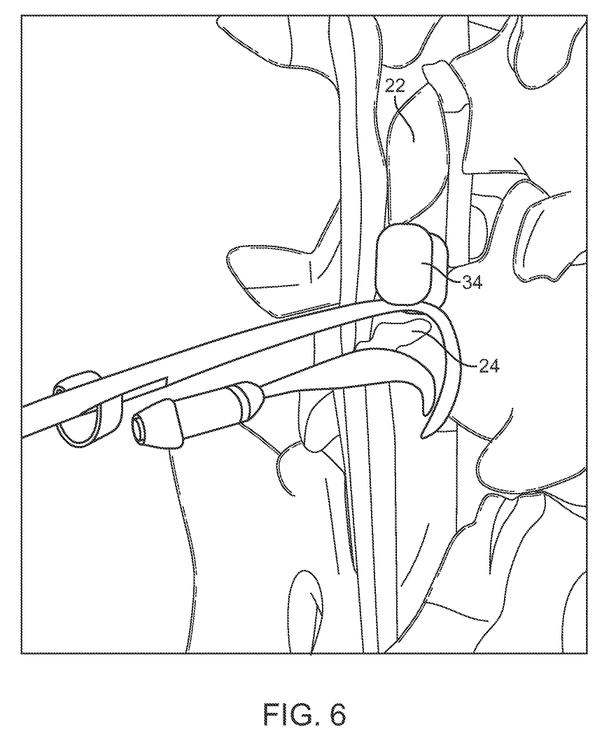

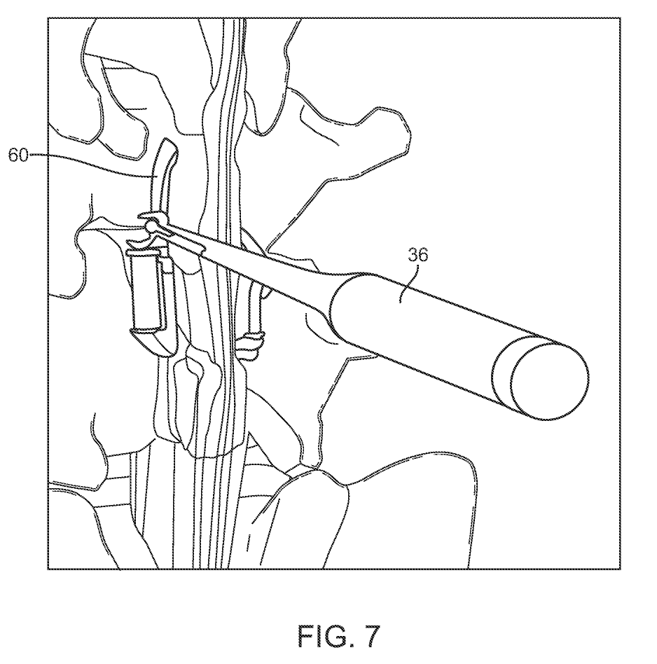

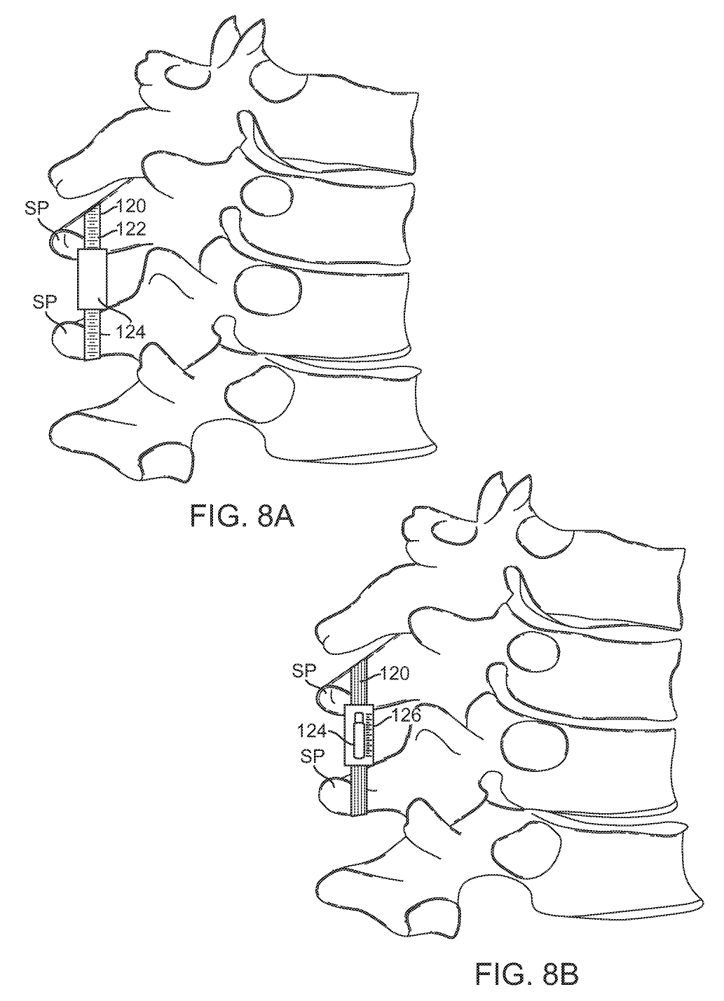

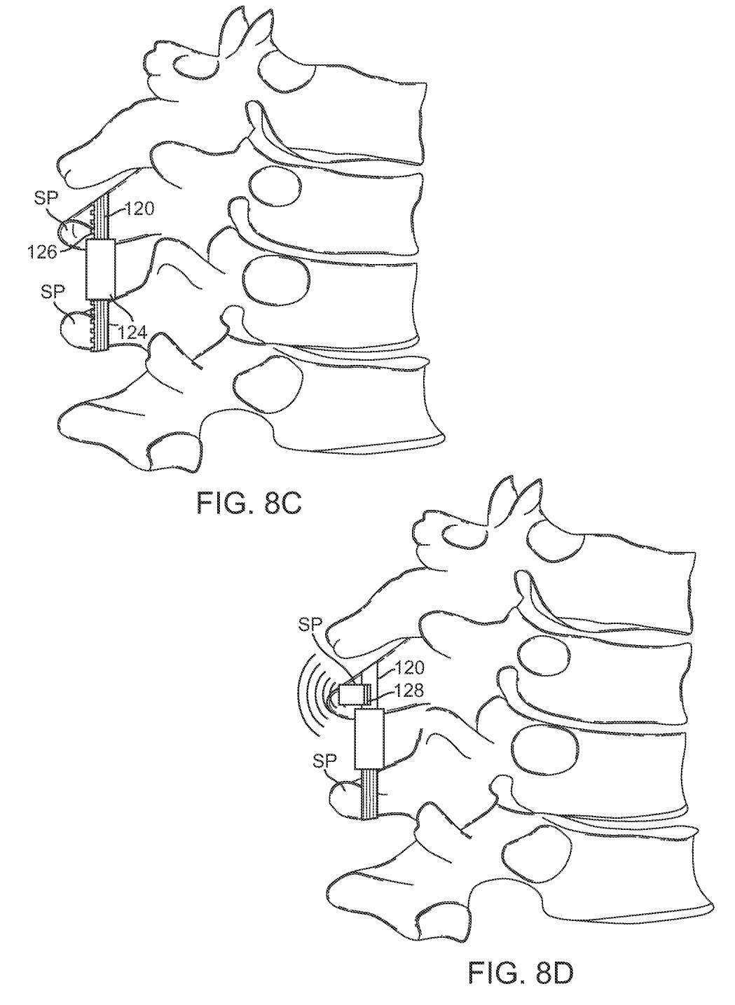

While tension or length may be adjusted in the tether structure by simply tightening the band around the spinous processes, the adjustment must also be carefully controlled so as not to over tighten or under tighten the tether structure. FIGS. 8A-8D illustrate several embodiments of tension control mechanisms that may be used. In FIG. 8A the tether structure 120 is disposed over spinous processes SP. Tether structure 120 comprises calibration marks 122 on the tether. A surgeon may adjust tension or length by tightening the tether structure 120 until a desired calibration mark is observed relative to the elastic member 124. The calibration marks 122 may also be used to measure the change in spacing between the spinous processes SP as adjustment occurs. Additionally, the calibration marks 122 may also be radiopaque and thus visible in a radiograph for post-operative evaluation. The tether structure is preferably compatible with magnetic resonance imaging (MRI) such that image quality is not significantly affected by the implant, nor does it result in excessive heating or dislodgement during MRI. FIG. 8B illustrates a tether structure 120 having a tension indicator gage 126. Tether structure 120 is wrapped around spinous processes SP and as tension is adjusted, gage 126 indicates deflection and/or force. FIG. 8C shows a tether structure 120 wrapped around spinous processes SP. An compliance member 124 controls elastic resistance of the tether structure 120. The tether structure 120 also has features 126 such as bumps or ridges that can be palpated through the patient's skin. This way, the surgeon can use tactile senses to determine the position of the compliance member 124 along the tether structure 120 during and after the procedure. FIG. 8D illustrates a tether structure around spinous processes SP. The tether structure 120 includes a strain gage 128 that can record stress and strain in the tether structure 120. Wireless technology similar to RFID technology or Blue Tooth technology may be used to transmit the stress/strain signal from gage 128 transcutaneously to a receiver or display unit.

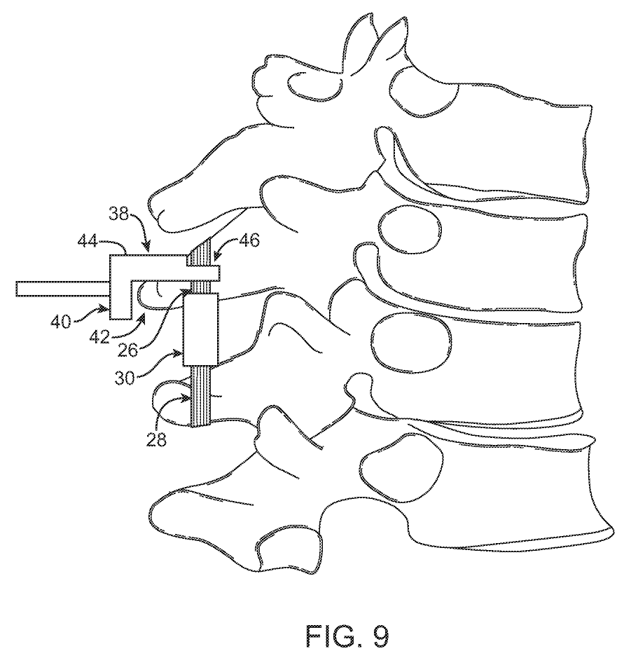

Referring now to FIG. 9, a positioning tool or jig 38 is optionally used to create an anterior-posterior axis to determine the target location at which to form the penetrations of the interspinous ligaments through which the tether structure will be advanced. The tool 38 has an end or stop 40 which engages the distal end 42 of the spinous process and a shaft or body 44 which defines a desired off set length and which has a location 46 for receiving and positioning the tool 20 and/or tether 26 of the implant. The tether may also be manually adjusted along the anterior-posterior axis, preferably so the tether is moved as far anterior as possible to prevent it from falling off the spinous process. Also, positioning the tether structure in the anterior-most position helps provide maximum elastic resistance during flexion.

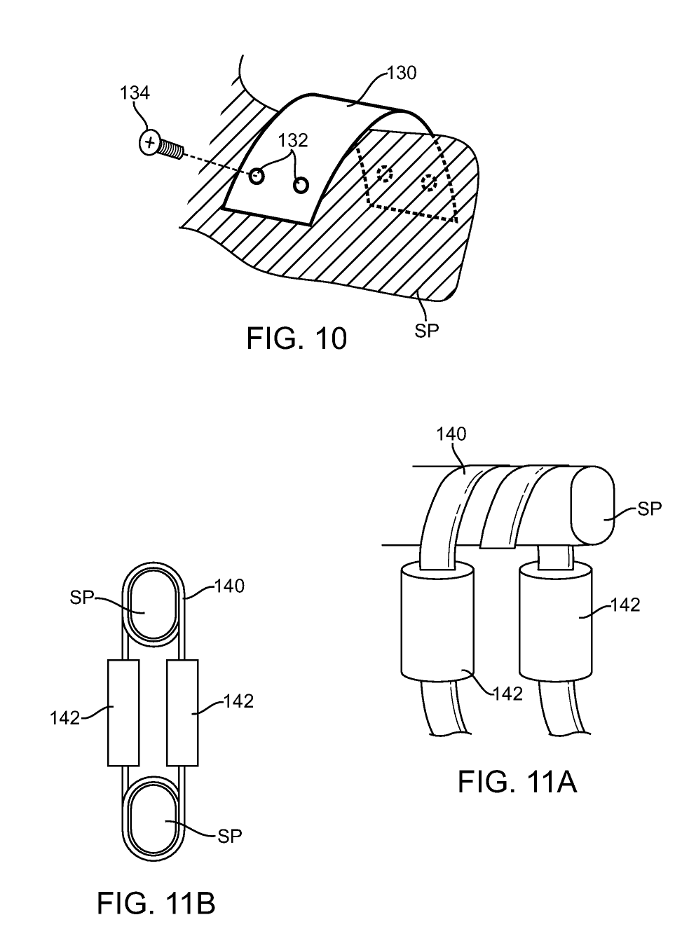

While anterior-posterior position of the tether structure on a spinous process will help keep the tether from falling off the spinous process, other coupling techniques may also be utilized. For example, in FIG. 10, a curved band 130 may be attached to the spinous process SP using a fixture such as a screw 134 through holes 132. The curved band may have an outer surface that adheres well to the tether structure to hold it in position, or the band 130 may be used to create a raised shoulder region that prevents posterior movement of the tether structure off of the spinous process SP. FIG. 11A shows a side view of how a tether structure 140 having elastic members 142 may be wrapped around a spinous process SP in order to help maintain the tether structure 140 on the spinous process. FIG. 11B shows an end view of the tether structure 140 wrapped around the spinous process SP. Additionally, penetrations may be placed in the spinous processes to help secure the tether. The tether may be threaded through the penetration or a fastener may be used to fix the tether to the bone. Also, the spinous process surfaces may be modified by drilling, notching, sanding, grinding or cutting to create a channel or region that holds the tether more effectively.

The embodiments discussed above are mainly directed at the treatment of degenerative disc disease, although they may also apply to other diseases. Degenerative spondylolisthesis is another disease that may benefit from the use of a tethering structure that restricts flexion of a spinal segment, especially when combined with other known spinal disorder treatments such as decompression and/or fusion.

Degenerative Spondylolisthesis (DS) is a common clinical condition that typically presents in the 5th to 8th decades. The listhesis, or anterior translation of the superior vertebra relative to the inferior vertebra, is associated with degenerative changes which make the facet joints less resistant to shear forces seen by the segment.

As the center of mass of the human body is almost always in front of the spine, there is typically a net shear force exerted on the spine during activities of daily living. Of the three joints that comprise the motion segment of every level of the spine (disc and two facet joints), the facet joints are most effective at resisting shear. As the facet joints degenerate, their typical coronal orientation becomes more sagittal, particularly in the superior section of the facet joint, further away from the pedicle. The facet joints' ability to resist shear decreases as they become more sagittally oriented. The typical finding on flexion/extension films in patients with degenerative spondylolisthesis is that the amount of anterior translation increases when the segment is in flexion, and decreases when the segment is in extension. In the extended position, more of the facet joint is engaged, and thus the overall resistance to shear is increased.

Patients with DS typically present with symptoms of stenosis, and these symptoms are relieved surgically with a decompression/laminectomy and fusion. Unfortunately, however, while decompression relieves pressure from nerves that cause pain, the removal of tissue involved in the decompression increases the flexion instability seen in DS, and, over time, the listhesis can increase and cause symptoms to recur. Because of the risk that a stand-alone decompression will increase post-operative instability, the standard of care in the United States is to treat degenerative spondylolisthesis patients with a decompression to treat the presenting symptoms and a fusion to prevent recurrence. The fusion may include instrumentation of the affected spinal segment including the use of pedicle screws and stabilization rods that have high morbidity and complication rates.

The use of a tether structure will allow the surgeon to perform a decompression to treat the presenting symptoms while maintaining the segment in an extended position. The tether structure will maintain the facets in the optimal position to resist shear and thus prevent progression of the anterior translation without requiring a fusion procedure.

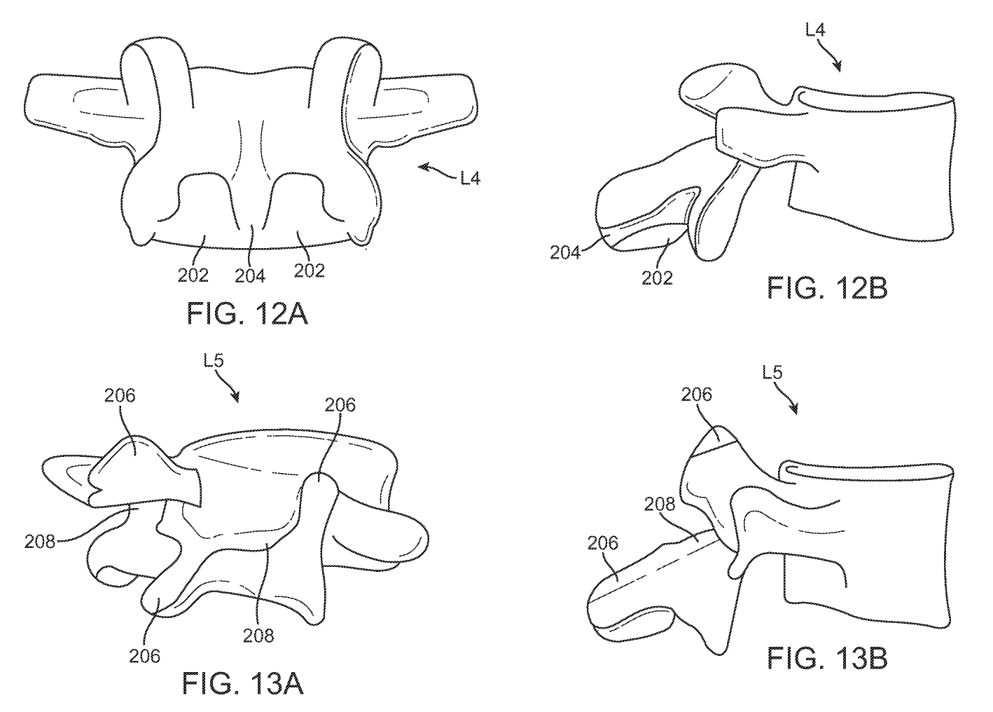

When treating DS, a surgeon performs decompression to relieve pressure on the nerve roots, typically at L4-L5, L3-L4, L5-S1, or elsewhere along the lumbar region of the spine. Bone is removed as required in order to provide pain relief, while still leaving some pieces of the bony structure intact. Often the superior portion of the superior spinous process in the affected spinal segment is left intact along with inferior portion of the inferior spinous process of the spinal segment. Additionally, a significant portion of the lamina will also be left intact. FIGS. 12A-12B illustrate typical areas of bone on L4 that may be removed during decompression. Regions 202 may be removed during a smaller decompression while in larger decompressions regions 202 and 204 may both be removed. FIG. 12A shows a posterior view of L4 and FIG. 12B shows a side view of L4. FIGS. 13A-13B illustrate typical areas of bone on L5 that may be removed during decompression. Regions 208 are removed in smaller decompressions and regions 206, 208 may both be removed in larger decompressions. FIG. 13A shows a perspective view of bone removal from L5 during decompression and FIG. 13B is a side view of L5 showing the bone removal regions.

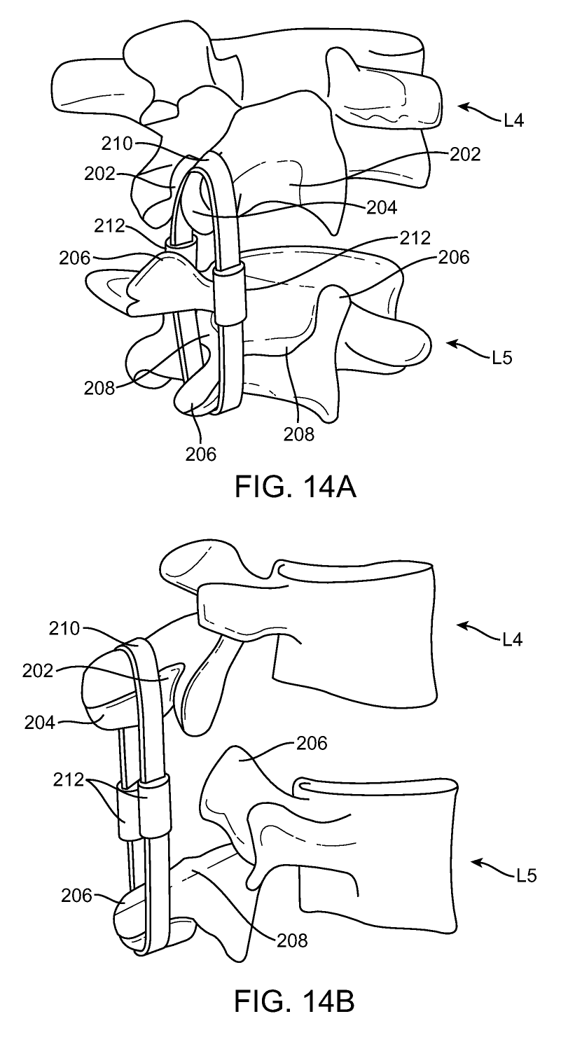

In an exemplary method of treating DS, bone decompression is performed at L4-L5 as described above with respect to FIGS. 12A-12B and FIGS. 13A-13B. A tether structure 210 is then disposed around an upper spinous process and a lower spinous process as seen in FIG. 14A. The tether structure may be any of the tether structures disclosed in this application and it is applied to the spinous processes in generally the same manner as previous discussed above with respect to FIGS. 4A-4D. The tether structure 210 includes compliance elements 212 that may be selected in order to adjust the elastic resistance of the tether structure 210. The elastic resistance should be high enough to provide a resistive force to flexion but not excessive, since this could result in damage to the surrounding spinous processes. In some cases it may be desirable for the tether structure to resist flexion with enough force to increase engagement of the facet joints during flexion. In still other cases, it may be desirable to tighten the tether structure over the spinous processes so that a relatively low finite force is applied even before flexion of a spinal segment from a neutral position. In other cases, it may be desirable to adjust the tether structure so that it applies a force to the spinous processes to create a new neutral position relative to the patient's pre-operative neutral position. FIG. 14A shows a posterior view of L4-L5 with the tether structure 210 applied to the spinous processes and the regions where bone removal may occur. FIG. 14B illustrates a side view of the tether structure 210 disposed around spinous processes in L4-L5. This procedure has a number of advantages over traditional methods for treating DS including requiring a smaller incision and being a less invasive. Also this procedure has less blood loss, requires less time and less anesthesia than traditional decompression/fusion surgery. Additionally, no fusion is performed and therefore there is no need for an autograft to be harvested from the patient. Also, pedicle screws are typically not required and therefore the patient has greater post-operative mobility and typically no risk of the complications and revisions associated with pedicle screws.

In other embodiments of a method for treating DS, a non-pedicle-screw based fusion may also be performed along with decompression. Non-pedicle-screw based fusions require the post-operative use of a lumbar brace for 3-6 months to ensure that the fusion has the best chance to heal, Even with the brace, the non-union rate still can be as high as about 40-50%. Bracing is not particularly effective in limiting segmental motion, and it is expensive and irritating for patients. Using a tether structure can replace the need for a postoperative brace by more effectively controlling segmental motion in these patients without significantly adding to the required soft tissue dissection or the length of the surgery. The tether's elastic construction limits the strains exerted on the spinous processes, minimizing the risk of fracture, especially in elderly patients with poor bone quality. The tether device furthermore avoids the potential mid- to long-term morbidity associated with the typical violation of the supradjacent facet joint associated with pedicle screw use, and may as such minimize the risk of the development of adjacent level syndromes. Therefore, a tether structure applied to an upper and a lower spinous process as previously described may provide a suitable internal brace to help the stabilize the treated spinal segment. Thus an external brace may not be required, eliminating the challenges of using such a brace, including patient discomfort, patient compliance as well as cost.

In some methods for treating stenosis with or without DS, a tether structure may also be applied to the spinous processes and the patient may also receive a discectomy or microdiscectomy and decompression.

In other methods for treating discogenic pain or degenerative disc disease, a tether structure may be applied to the spinous processes in a patient also receiving a discectomy or microdiscectomy. Often such a microdiscectomy is performed to remove material after the herniation of a disc. The tether structure in this context would chronically decrease the loads on the disc after the microdiscectomy, potentially relieving pain and potentially decreasing the risk of recurrence of herniation. In this context, the tether structure might optionally be used together with other treatments of the disc or annulus that are intended to reduce re-herniation risk, such as placing or injecting a sealant into the annulus or nucleus, implanting a barrier device in the annulus, or suturing or repairing the annulus.

Furthermore, in all embodiments, tether structure tension or length may be adjusted post-procedure in order to optimize performance of the tether structure after it has been implanted. Such post-procedure adjustment may be accomplished via wireless technology that communicates between an external device and a component on the tether structure in order to adjust the tether structure. In some embodiments, such technology would allow patients to self-adjust properties of the implanted tether structure, such as length, stiffness, or tension to accommodate a patient's physical characteristics and needs.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for constraining spinous processes to elastically limit flexion of a spinal segment, said method comprising:
piercing an interspinous ligament to form a first penetration above an upper side of an upper spinous process;
advancing a first end of a first tether through the first penetration;
piercing the interspinous ligament to form a second penetration below a lower side of a lower spinous process;
advancing a second end of a second tether through the second penetration;
joining the first and second tethers to form an extensible tether structure wherein the structure couples the upper and lower spinous processes together while permitting extension therebetween; and
adjusting the tether structure so as to set a relative distance or angle between the upper and lower spinous processes to a target value.

2. A method as in claim 1, wherein a single tether comprising the first and second tethers is advanced through the first and second penetrations and joined to form the tether structure.

3. A method as in claim 1, further comprising joining the first and second tethers together at another point.

4. A method as in claim 1, wherein the first tether is advanced through the first penetration from a first side of the spinal segment and the second tether is advanced through the second penetration from a second side of the spinal segment, the first and second sides being on opposite sides of the spinal segment midline.

5. A method as in claim 1, wherein the first and second tethers are joined outside a patient's body.

6. A method as in claim 1, wherein the supraspinous ligament or the multifidus tendon in the spinal segment remain intact.

7. A method as in claim 1, wherein the portion of the interspinous ligament disposed between the upper and lower spinous processes remains intact.

8. A method as in claim 1, wherein at least one intermediate spinous process is disposed between the upper and lower spinous processes.

9. A method as in claim 1, further comprising forming a penetration through either the upper or lower spinous process.

10. A method as in claim 1, further comprising modifying a surface of either the upper or lower spinous process.

11. A method as in claim 10, wherein modifying the surface comprises at least one of cutting, sanding, grinding, drilling and notching.

12. A method as in claim 1, further comprising positioning the tether structure along either the upper or lower spinous process along a generally anterior-posterior axis.

13. A method as in claim 1, further comprising looping the tether structure around either the upper or lower spinous process at least 360 degrees.

14. A method as in claim 1, wherein advancing either the first or second tether ends comprises pushing either end through the first or second penetration.

15. A method as in claim 14, wherein either the first or second tether end is pushed through the first or second penetration during formation thereof.

16. A method as in claim 1, wherein advancing either the first or second tether ends comprises pulling either end through the first or second penetration.

17. A method as in claim 1, wherein adjusting comprises adjusting tension in the tether structure.

18. A method as in claim 1, wherein adjusting comprises adjusting length of the tether structure.

19. A method as in claim 1, further comprising placing a temporary spacer between the upper and lower spinous processes so as to fix distance therebetween without disrupting the interspinous ligament.

20. A method as in claim 1, wherein adjusting the tether structure comprises observing visual indicators thereon.

21. A method as in claim 1, wherein adjusting the tether structure comprises changing a compliance element in the tether structure to change the elastic resistance of the tether structure.

22. A method as in claim 1, wherein adjusting the tether structure comprises holding the upper and lower spinous processes at a fixed distance from each other while changing tether structure length.

23. A method as in claim 1, wherein adjusting the tether structure comprises changing effective length of the tether structure, the effective length being that portion of the tether structure that is continuous.

24. A method as in claim 1, wherein adjusting the tether structure comprises measuring landmarks on the spinal segment.

25. A method as in claim 24, wherein the landmarks comprise points on the upper and lower spinous processes.

26. A method as in claim 1, wherein the target value is determined while the spinal segment is disposed in a neutral position.

27. A method as in claim 1, wherein the target value is determined while the patient is in a relaxed standing position.

28. A method as in claim 1, further comprising expanding the first and second penetrations prior to advancing the first and second ends therethrough.

29. A method as in claim 1, further comprising severing the tether structure to remove excess material therefrom.

30. A method as in claim 1, further comprising joining additional components with the tether structure.

31. A method as in claim 30, wherein the additional components are selected from compliance members, extension members, compression members, tension members, attachment buckles, and adjustment members.

32. A method as in claim 1, further comprising re-adjusting tension or length in the tether structure after the first adjustment.

33. A method for treating degenerative spondylolisthesis, said method comprising:
decompressing the spinal segment;
creating a pathway across a midline of the spinal segment above an upper side of an upper spinous process;
advancing a first end of a first tether along the pathway;
creating a second pathway across the midline of the spinal segment below a lower side of a lower spinous process;
advancing a second end of a second tether along the second pathway;
joining the first and second tethers to form an extensible tether structure wherein the structure couples the upper and lower spinous processes together thereby elastically limiting flexion of a spinal segment containing the upper and lower spinous processes while permitting extension therebetween; and
adjusting the tether structure so as to set a relative distance or angle between the upper and lower spinous processes to a target value.

34. A method as in claim 33, wherein a single tether comprising the first and second tethers is joined to form the tether structure.

35. A method as in claim 33, further comprising joining the first and second tethers together at another point.

36. A method as in claim 33, wherein the first tether is advanced through a first side of the spinal segment and the second tether is advanced through a second side of the spinal segment, the first and second sides being on opposite sides of the spinal segment midline.

37. A method as in claim 33, wherein the first and second tethers are joined outside a patient's body.

38. A method as in claim 33, wherein the supraspinous ligament or the multifidus tendon in the spinal segment remain intact.

39. A method as in claim 33, wherein the portion of the interspinous ligament disposed between the upper and lower spinous processes remains intact.

40. A method as in claim 33, wherein at least one intermediate spinous process is disposed between the upper and lower spinous processes.

41. A method as in claim 33, further comprising forming a penetration through either the upper or lower spinous process.

42. A method as in claim 33, further comprising modifying a surface of either the upper or lower spinous process.

43. A method as in claim 42, wherein modifying the surface comprises at least one of cutting, sanding, grinding, drilling and notching.

44. A method as in claim 33, further comprising positioning the tether structure along either the upper or lower spinous process along a generally anterior-posterior axis.

45. A method as in claim 33, further comprising looping the tether structure around either the upper or lower spinous process at least 360 degrees.

46. A method as in claim 33, wherein advancing either the first or second tether ends comprises pushing either end along either pathway.

47. A method as in claim 33, wherein advancing either the first or second tether ends comprises pulling either end along either pathway.

48. A method as in claim 33, wherein adjusting comprises adjusting tension in the tether structure.

49. A method as in claim 33, wherein adjusting comprises adjusting length of the tether structure.

50. A method as in claim 33, further comprising placing a temporary spacer between the upper and lower spinous processes so as to fix distance therebetween without disrupting the interspinous ligament.

51. A method as in claim 33, wherein adjusting the tether structure comprises observing visual indicators thereon.

52. A method as in claim 33, wherein adjusting the tether structure comprises changing a compliance element in the tether structure to change the elastic resistance of the tether structure.

53. A method as in claim 33, wherein adjusting the tether structure comprises holding the upper and lower spinous processes at a fixed distance from each other while changing tether structure length.

54. A method as in claim 33, wherein adjusting the tether structure comprises changing effective length of the tether structure, the effective length being that portion of the tether structure that is continuous.

55. A method as in claim 33, wherein adjusting the tether structure comprises measuring landmarks on the spinal segment.

56. A method as in claim 55, wherein the landmarks comprise points on the upper and lower spinous processes.

57. A method as in claim 33, wherein the target value is determined while the spinal segment is disposed in a neutral position.

58. A method as in claim 33, wherein the target value is determined while the patient is in a relaxed standing position.

59. A method as in claim 33, further comprising expanding the pathway or the second pathway prior to advancing the first and second tethers therealong.

60. A method as in claim 33, further comprising severing the tether structure to remove excess material therefrom.

61. A method as in claim 33, further comprising joining additional components with the tether structure.

62. A method as in claim 61, wherein the additional components are selected from compliance members, extension members, compression members, tension members, attachment buckles, and adjustment members.

63. A method as in claim 33, further comprising re-adjusting tension or length in the tether structure after the first adjustment.

64. A method as in claim 33, wherein adjusting the tether structure applies a force to the spinous processes to create a new neutral position relative to the pre-operative neutral position.

65. A method as in claim 33, wherein adjusting the tether structure subsequently reduces translation of the upper spinous process relative to the lower spinous process along an anterior-posterior axis due to shear forces in the spinal segment.

66. A method as in claim 33, wherein decompressing the spinal segment comprises removing bone, disc or ligament therefrom.

67. A method as in claim 33, wherein creating a pathway comprises piercing the interspinous ligament.

68. A method as in claim 33, wherein creating a second pathway comprises piercing the interspinous ligament.

69. A method for treating a spinal disorder, said method comprising:
decompressing the spinal segment;
creating a pathway across a midline of the spinal segment above an upper side of an upper spinous process;
advancing a first end of a first tether along the pathway;
creating a second pathway across the midline of the spinal segment below a lower side of a lower spinous process;
advancing a second end of a second tether along the second pathway;
joining the first and second tethers to form an extensible tether structure wherein the structure couples the upper and lower spinous processes together thereby elastically limiting flexion of a spinal segment containing the upper and lower spinous processes while permitting extension therebetween;
measuring tension in the tether structure; and
adjusting the tether structure so as to set the tension to a target value.
